(19) United States
(12) Patent Application Publication
(10) Pub. No.: US 2012/0078111 A1
(43) Pub. Date: Mar. 29, 2012

(54) ULTRASOUND PROBE

(75) Inventors: Tsuyoshi TANABE, Ashigara-kami-gun (JP); Masafumi NOGUCHI, Ashigara-kami-gun (JP)

(73) Assignee: FUJIFILM Corporation, Tokyo (JP)

(21) Appl. No.: 13/247,297

(22) Filed: Sep. 28, 2011

(30) Foreign Application Priority Data
Sep. 29, 2010 (JP) 2010-219311
Sep. 29, 2010 (JP) 2010-219723

(51) Int. Cl. A61B 8/14

(52) U.S. Cl. 600/459

(57) ABSTRACT

An ultrasound probe comprises: a transducer array having an ultrasound transmission/reception surface; a housing for containing a transducer array, a transmission/reception board, a wireless communication board and a battery; and a puncture guide fixed to the housing and having a puncture needle insertion port located on a line extending in a direction of arrangement of the transducer array, wherein when the ultrasound transmission/reception surface of the transducer array is in a horizontal posture, the probe has a center of gravity lying between a center of arrangement of the transducer array and the puncture needle insertion port of the puncture guide in the direction of arrangement of the transducer array and, at the same time, being almost in accordance with a centerline of arrangement of the transducer array in a direction orthogonal to the direction of arrangement of the transducer array.

### BACKGROUND OF THE INVENTION

The present invention relates to ultrasound probes, particularly those having puncture guides.

The present invention also relates to ultrasound probes for use in diagnosis by elasticity imaging.

Conventionally, ultrasound diagnostic apparatus using ultrasound images have been put to use in the medical field. In general, this type of ultrasound diagnostic apparatus comprises an ultrasound probe having a built-in transducer array, and an apparatus body connected to the ultrasound probe. The ultrasound probe transmits ultrasonic waves toward a subject, receives ultrasonic echoes from the subject, and the apparatus body electrically processes the reception signals to generate an ultrasound image.

In a common treatment using such an ultrasonic diagnostic apparatus as above, an operator holds the ultrasound probe and makes it into contact with a subject with one hand to detect a specified site in the subject and, concurrently, collects cells or tissues, or administers a medicament by performing a puncturing maneuver with the other hand. An ultrasonic diagnosis involving a puncturing maneuver needs to be conducted so that the inserted puncture needle may reliably reach a target site in a subject, or, the puncture needle may not become fuzzy or even be lost in a tomographic image. Such a diagnosis is hard to carry out without some degree of experience or skill.

On the apparatus as disclosed in JP 2006-87659 A, for instance, a wired ultrasound probe with a puncture adaptor attached thereto is used to insert a puncture needle supported by the puncture adaptor into the living body from the vicinity of an edge of the prove face made into contact with the living body.

If the puncture adaptor as described in JP 2006-87659 A is to be attached to an ultrasound probe of a wireless type, however, more skill will be required in order to hold the ultrasound probe with one hand and, concurrently, perform a puncturing maneuver with the other hand to make measurements with precision and safely because the wireless ultrasound probe has various boards, a battery, and so forth contained in a housing, so that it is generally heavier than such a wired ultrasound probe as described in JP 2006-87659 A.

Diagnosis by elasticity imaging (elastography) for measuring a local stiffness of a tissue in a subject is one of the diagnoses using an ultrasonic diagnostic apparatus. In an exemplary diagnosis by elasticity imaging described in WO 2005/120358 A, pressure is applied to the region of interest in a subject by pressing an ultrasound probe with a pressure plate attached thereto against the surface of the subject and, based on the data on the stress distribution and the distortion thus obtained, information on the elasticity of the region of interest is imaged. Application of pressure to the region of interest is carried out by holding the ultrasound probe with one hand, and pressing the pressure plate against the surface of the subject in a direction perpendicular to the surface. The pressure plate is preferably pressed against the subject so that a uniform pressure may be applied to the region of interest.

The stiffness measurement by pressing a probe against a subject as mentioned above is hard to perform without some degree of experience or skill. Particularly if a wireless ultrasound probe is to be used instead of such a conventional, wired ultrasound probe as described in WO 2005/120358 A, more skill will be required in order to keep the pressure plate in a perpendicular contact with the surface of a subject and press the pressure plate so that a uniform pressure may be applied to the area of the subject surface that is in contact with the plate, so as to conduct a stable and accurate diagnosis by elasticity imaging because the wireless ultrasound probe has various boards, a battery, and so forth contained in a housing, so that it is generally heavier than a conventional, wired ultrasound probe.

### SUMMARY OF THE INVENTION

An object of the present invention is to solve the above problems with the prior art to provide a wireless ultrasound probe allowing a stable ultrasonic diagnosis involving a puncturing maneuver.

Another object of the present invention is to provide a wireless ultrasound probe allowing a stable diagnosis by elasticity imaging.

An ultrasound probe according to a first aspect of the present invention comprises:

a transducer array having an ultrasound transmission/reception surface;

a transmission/reception board including a transmission/reception circuit for the transducer array;

a wireless communication board having a wireless communication circuit mounted thereon for wireless communication with an external communication device;

a battery for supplying power to the transmission/reception circuit and the wireless communication circuit;

a housing for containing the transducer array, the transmission/reception board, the wireless communication board and the battery; and

a puncture guide fixed to the housing and having a puncture needle insertion port located on a line extending in a direction of arrangement of the transducer array,

wherein when the ultrasound transmission/reception surface of the transducer array is in a horizontal posture, the probe has a center of gravity lying between a center of arrangement of the transducer array and the puncture needle insertion port of the puncture guide in the direction of arrangement of the transducer array and, at the same time, being almost in accordance with a centerline of arrangement of the transducer array in a direction orthogonal to the direction of arrangement of the transducer array.

An ultrasound probe according to a second aspect of the present invention comprises:

a transducer array having an ultrasound transmission/reception surface;

a transmission/reception board including a transmission/reception circuit of the transducer array;

a wireless communication board having a wireless communication circuit mounted thereon for wireless communication with an external communication device;

a battery for supplying power to the transmission/reception circuit and the wireless communication circuit;

a housing for containing the transducer array, the transmission/reception board, the wireless communication board and the battery; and

a pressure plate fixed to the housing and having a pressure face almost flush with the ultrasound transmission/reception surface of the transducer array,

wherein when the ultrasound transmission/reception surface of the transducer array is in a horizontal posture, the probe has a center of gravity almost in accordance with a centerline of arrangement of the transducer array in both a direction of arrangement of the transducer array and a direction orthogonal to the direction of arrangement of the transducer array.

### BRIEF DESCRIPTION OF THE DRAWINGS

In the accompanying drawings:

FIG. 1A is a front view of an ultrasound probe according to Embodiment 1 of the present invention, and FIG. 1B is a side view thereof;

FIG. 2 is a block diagram showing the inner configuration of an ultrasonic diagnostic apparatus provided with the ultrasound prove according to Embodiment 1;

FIG. 3A is a front view of an ultrasound probe according to Embodiment 2, and FIG. 3B is a side view thereof;

FIG. 4 is a front view of an ultrasound probe according to Embodiment 3;

FIG. 5A is a front view of an ultrasound probe according to Embodiment 4, and FIG. 5B is a side view thereof;

FIG. 6 is a block diagram showing the inner configuration of an ultrasonic diagnostic apparatus provided with the ultrasound prove according to Embodiment 4;

FIG. 7A is a front view of an ultrasound probe according to Embodiment 5, and FIG. 7B is a side view thereof;

FIG. 8 is a front view of an ultrasound probe according to Embodiment 6;

FIG. 9A is a diagram depicting a weight used in an ultrasound probe according to Embodiment 7;

FIG. 9B is a diagram depicting a weight used in an ultrasound probe according to a modification of Embodiment 7; and

FIG. 10 is a partial sectional view of an ultrasound probe according to Embodiment 8.

### DETAILED DESCRIPTION OF THE INVENTION

In the following, embodiments of the present invention are illustrated in reference to the accompanying drawings.

### Embodiment 1

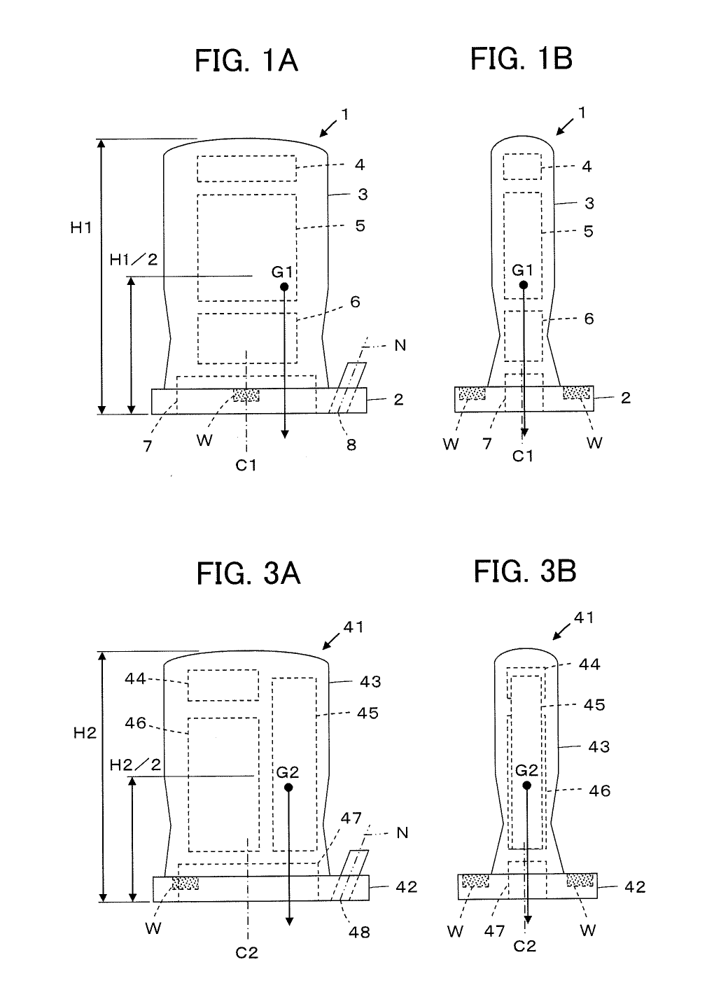

FIGS. 1A and 1B show an ultrasound probe according to Embodiment 1. The ultrasound probe includes a probe body 1, a puncture guide 2 fixed to the probe body 1, and a pair of weights W mounted on the puncture guide 2.

The probe body 1 has a housing 3, and a wireless communication board 4, a battery 5, a transmission/reception board 6, and a transducer unit 7 including a transducer array are contained in the housing 3.

The puncture guide 2 is integrally fixed to the housing 3 of the probe body 1, and has a puncture needle insertion port 8 for a puncture needle N, with the port 8 being located on a line extending in the direction of arrangement of the transducer array in the transducer unit 7. In the top face of the puncture guide 2, the weights W are embedded so that one weight W may be located on each side of the transducer array in a direction orthogonal to the direction of arrangement of the transducer array.

The wireless communication board 4, the battery 5, and the transmission/reception board 6 are each positioned on a centerline of arrangement C1 of the transducer array.

The transducer unit 7 is a transducer unit comprising a transducer array, an acoustic matching layer, an acoustic lens, a backing layer, and the like.

As seen from FIGS. 1A and 1B, the ultrasound probe as shown has a center of gravity G1 lying between the centerline of arrangement C1 of the transducer array and the puncture needle insertion port 8 of the puncture guide 2 in the direction of arrangement of the transducer array and, at the same time, being almost in accordance with the centerline of arrangement C1 of the transducer array in the direction orthogonal to the direction of arrangement of the transducer array when an ultrasound transmission/reception surface of the transducer array is in a horizontal posture.

With the ultrasound probe having such a structure as above, an operator is able to keep the probe with one hand in a stable posture with respect to a subject and, accordingly, perform a puncturing maneuver accurately and easily with the other hand.

As shown in FIG. 1A, the center of gravity G1 is at a height under H1/2, one half the height of the housing 3 which is H1, when the ultrasound transmission/reception surface of the transducer array is in a horizontal posture.

In the ultrasound probe, the transducer unit 7 generally needs to be positioned at the bottom of the housing 3. Since it is desirable to position the transmission/reception board 6 for driving the transducer unit 7 close to the transducer unit 7, the battery 5 with a large weight is inevitably placed in the housing 3 in an upper position, which may cause the center of gravity G1 of the probe to lie in an upper position, leading to a reduction in stability. In the ultrasound probe according to Embodiment 1, however, the center of gravity G1 lies in the lower half of the probe owing to the weights W embedded in the puncture guide 2, so that an operator is able to manipulate the probe in a more stable posture.

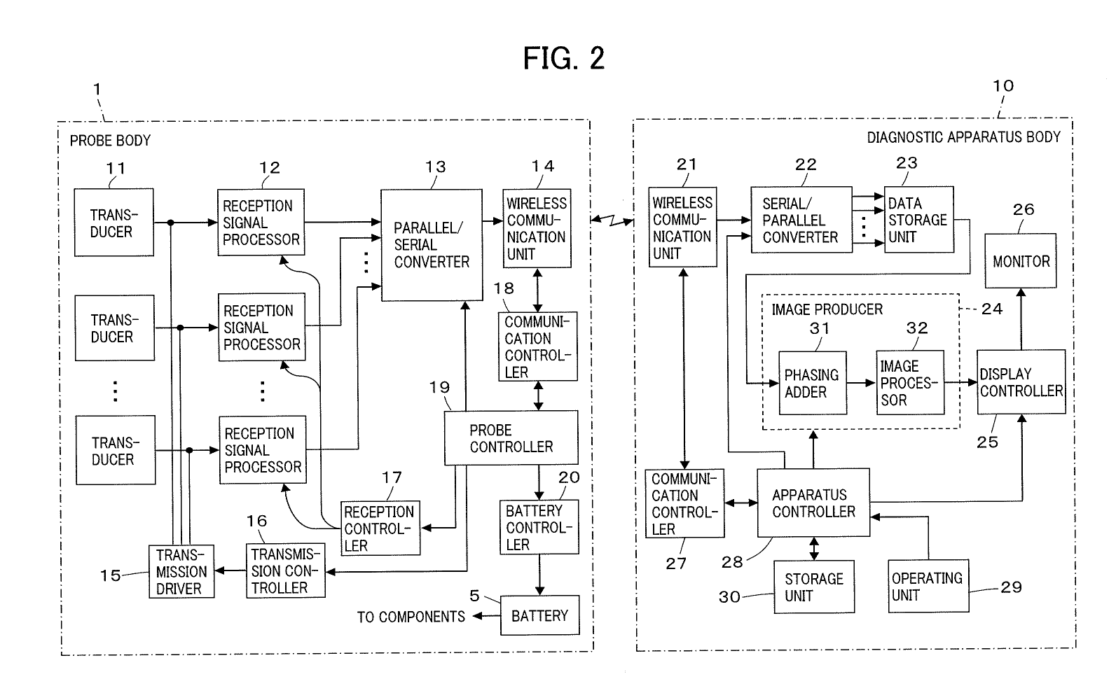

FIG. 2 shows the configuration of an ultrasonic diagnostic apparatus provided with the ultrasound probe according to Embodiment 1. A diagnostic apparatus body 10 is connected to the probe body 1 as an external communication device through wireless communication.

The probe body 1 includes a plurality of ultrasound transducers 11 constituting a one-dimensional or two-dimensional transducer array, and the transducers 11 are connected to corresponding reception signal processors 12, respectively, which in turn are connected to a wireless communication unit 14 via a parallel/serial converter 13. The transducers 11 are connected to a transmission controller 16 via a transmission driver 15, and the reception signal processors 12 are connected to a reception controller 17, while the wireless communication unit 14 is connected to a communication controller 18. The parallel/serial converter 13, the transmission controller 16, the reception controller 17, and the communication controller 18 are connected to a probe controller 19.

The probe controller 19 is connected to the battery 5 via a battery controller 20.

The transducers 11 each transmit ultrasonic waves according to driving signals supplied from the transmission driver 15 and receive ultrasonic echoes from the subject to output reception signals. Each of the transducers 11 comprises a piezoelectric element composed of a piezoelectric ceramic or monocrystal represented by PZT (lead zirconate titanate), a piezoelectric polymer represented by a PVDF (polyvinylidene fluoride), or other piezoelectric material, and electrodes provided at both ends of the piezoelectric element.

When the electrodes of each of such transducers 11 are supplied with a voltage, which may be in the form of pulse or continuous waves, the piezoelectric element expands and contracts, and the transducer 11 generates ultrasonic waves in the form of pulse or continuous waves. These ultrasonic waves are synthesized to form an ultrasonic beam. As each transducer 11 receives propagating ultrasonic waves, it expands and contracts to generate an electric signal, and outputs the electric signal as a reception signal for ultrasonic waves.

The transmission driver 15 comprises, for example, a plurality of pulsers, and adjusts the delay amounts of driving signals for the individual transducers 11 based on a transmission delay pattern selected by the transmission controller 16 so that the ultrasonic waves transmitted from the transducers 11 may form a broad ultrasonic beam to cover an area of a tissue in a subject, then supplies the transducers 11 with the adjusted driving signals.

Under the control of the reception controller 17, the reception signal processor 12 on each channel subjects the reception signal outputted from the corresponding transducer 11 to quadrature detection or quadrature sampling process to produce a complex base band signal and samples the complex base band signal to generate sample data containing information on the area of the tissue. The reception signal processors 12 may generate sample data by performing data compression for high-efficiency coding on the data obtained by sampling the complex base band signals.

The parallel/serial converter 13 converts parallel sample data generated by the reception signal processors 12 on a plurality of channels into serial sample data.

The wireless communication unit 14 performs carrier modulation according to the serial sample data to generate a transmission signal and supplies an antenna with the transmission signal so that the antenna may transmit radio waves to achieve transmission of the sample data. The modulation methods that may be employed herein include ASK (Amplitude Shift Keying), PSK (Phase Shift Keying), QPSK (Quadrature Phase Shift Keying), and 16QAM (16 Quadrature Amplitude Modulation).

The wireless communication unit 14 transmits the sample data to the diagnostic apparatus body 10 and receives various control signals from the diagnostic apparatus body 10 through wireless communication with the diagnostic apparatus body 10, and outputs the received control signals to the communication controller 18. The communication controller 18 controls the wireless communication unit 14 so that the sample data may be transmitted with a transmission field intensity set by the probe controller 19, and outputs various control signals received by the wireless communication unit 14 to the probe controller 19.

The probe controller 19 controls various components of the probe body 1 according to various control signals transmitted from the diagnostic apparatus body 10.

The battery 5 functions as a power supply unit of the probe body 1 for supplying power to the components in the probe body 1 that are in need of power. The battery controller 20 controls the power supply from the battery 5 to the components in the probe body 1.

The probe body 1 uses a scan method of linear scan type, sector scan type, or the like.

On the other hand, the diagnostic apparatus body 10 includes a wireless communication unit 21, which is connected to a data storage unit 23 via a serial/parallel converter 22. The data storage unit 23 is connected to an image producer 24. The image producer 24 is connected to a monitor 26 via a display controller 25. The wireless communication unit 21 is also connected to a communication controller 27, and the serial/parallel converter 22, the image producer 24, the display controller 25, and the communication controller 27 are connected to an apparatus controller 28. The apparatus controller 28 is connected to an operating unit 29 for an operator to perform input operations and a storage unit 30 for storing operation programs.

The wireless communication unit 21 transmits various control signals to the probe body 1 through wireless communication with the probe body 1. In addition, the wireless communication unit 21 demodulates a signal received by an antenna to output serial sample data.

The communication controller 27 controls the wireless communication unit 21 so that various control signals may be transmitted with a transmission field intensity set by the apparatus controller 28.

The serial/parallel converter 22 converts the serial sample data outputted from the wireless communication unit 21 into parallel sample data. The data storage unit 23 comprises a memory, a hard disk or the like, and stores the sample data as converted by the serial/parallel converter 22 for at least one frame.

The image producer 24 performs reception focusing process on the sample data as read out per frame from the data storage unit 23 to generate an image signal representing a diagnostic ultrasound image. The image producer 24 includes a phasing adder 31 and an image processor 32.

The phasing adder 31 selects one reception delay pattern from the previously stored reception delay patterns according to the reception direction set in the apparatus controller 28 and, based on the selected reception delay pattern, provides the complex base band signals represented by the sample data with their respective delays before adding them to perform the reception focusing process. By this reception focusing, a base band signal (sound ray signal) where the ultrasonic echoes are well focused is generated.

The image processor 32 generates a B-mode image signal, which is tomographic image information on the tissue in the subject, according to the sound ray signal generated by the phasing adder 31. The image processor 32 includes an STC (sensitivity time control) and a DSC (digital scan converter). For the sound ray signal, the STC corrects attenuation due to distance according to the depth of the reflection position of the ultrasonic waves. The DSC converts the sound ray signal corrected by the STC into an image signal compatible with a common television signal scanning method (raster conversion), and performs required image processing, such as gradation processing, to generate a B-mode image signal.

The display controller 25 causes the monitor 26 to display a diagnostic ultrasound image according to the image signal generated by the image producer 24. The monitor 26 includes a display device such as an LCD, and displays a diagnostic ultrasound image under the control of the display controller 25.

The apparatus controller 28 controls various components in the diagnostic apparatus body 10 according to various control signals and the like transmitted from the probe body 1.

In the diagnostic apparatus body 10 as above, the serial/parallel converter 22, the image producer 24, the display controller 25, the communication controller 27, and the apparatus controller 28 are implemented by a CPU associated with operation programs for giving the CPU instructions on various kinds of processing, while the above elements may also be implemented by a digital circuitry. The operation programs are stored in the storage unit 30. The storage unit 30 may include as a recording medium a flexible disk, MO, MT, RAM, CD-ROM, DVD-ROM or the like besides a built-in hard disk.

In the probe body 1, the wireless communication unit 14 and the communication controller 18 are mounted on the wireless communication board 4, and the reception signal processors 12, the parallel/serial converter 13, the transmission driver 15, the transmission controller 16, the reception controller 17, the probe controller 19, and the battery controller 20 are mounted on the transmission/reception board 6.

The ultrasound probe according to Embodiment 1 is not particularly limited in weight. As an example, the ultrasound probe is such that the housing 3 is 40 g, the wireless communication board 4 is 10 g, the battery 5 is 40 g, the transmission/reception board 6 is 30 g, the transducer unit 7 is 20 g, the puncture guide 2 is 40 g, and the weights W are 7 g each in weight.

The ultrasound probe of Embodiment 1 operates as follows.

During an ultrasonic diagnosis involving a puncturing maneuver, an operator initially holds the probe body 1 with one hand to bring the puncture guide 2 into contact with the surface of a subject, then takes the puncture needle N with the other hand to perform the puncturing maneuver. The transducers 11 transmit ultrasonic waves according to the driving signals as supplied from the transmission driver 15 of the prove body 1, and receive ultrasonic echoes from the subject before they output reception signals to the corresponding reception signal processors 12, respectively. The sample data as generated by the reception signal processors 12 supplied with the reception signals are converted by the parallel/serial converter 13 into serial data, then transmitted from the wireless communication unit 14 to the diagnostic apparatus body 10 through wireless communication. The sample data as received by the wireless communication unit 21 of the diagnostic apparatus body 10 are converted by the serial/parallel converter 22 into parallel data, and stored in the data storage unit 23. Subsequently, the sample data are read out per frame from the data storage unit 23, an image signal is generated by the image producer 24, and a diagnostic ultrasound image is displayed on the monitor 26 by the display controller 25 according to the image signal.

By using the ultrasound probe of Embodiment 1, the puncture guide 2 as pressed against a subject can be kept in a stable posture with respect to the subject when an operator performs a puncturing maneuver because the center of gravity G1 of the probe lies between the centerline of arrangement C1 of the transducer array and the puncture needle insertion port 8 of the puncture guide 2 in the direction of arrangement of the transducer array and, at the same time, is almost in accordance with the centerline of arrangement C1 of the transducer array in the direction orthogonal to the direction of arrangement of the transducer array.

Since the weights W are arranged so that one weight W may be located on each side of the transducer array in the direction orthogonal to the direction of arrangement of the transducer array, the tilting in a plane perpendicular to the direction of arrangement of the transducer array, so-called “swing,” hardly occurs, which also makes it possible to keep the puncture guide 2 in a stable posture, and thereby conduct an ultrasonic diagnosis while performing a puncturing maneuver accurately and easily.

Moreover, the weights W embedded in the puncture guide 2 allow the center of gravity G1 to be present at a height under H1/2, one half the height of the housing 3 which is H1, when the ultrasound transmission/reception surface of the transducer array is in a horizontal posture, so that the ultrasound probe is manipulated in a more stable posture.

In Embodiment 1, the weights W are embedded in the top face of the puncture guide 2, although not limitedly. The weights W may be embedded in the bottom face of the puncture guide 2.

### Embodiment 2

FIGS. 3A and 3B show an ultrasound probe according to Embodiment 2. The ultrasound probe includes a probe body 41, a puncture guide 42 fixed to the probe body 41, and a pair of weights W mounted on the puncture guide 42.

The probe body 41 has a housing 43, and a wireless communication board 44, a battery 45, a transmission/reception board 46, and a transducer unit 47 including a transducer array are contained in the housing 43.

The puncture guide 42 is integrally fixed to the housing 43 of the probe body 41, and has a puncture needle insertion port 48 located on a line extending in the direction of arrangement of the transducer array in the transducer unit 47.

The wireless communication board 44, the battery 45, the transmission/reception board 46, and the transducer array 47 are identical in structure and function to the wireless communication board 4, the battery 5, the transmission/reception board 6, and the transducer array 7 of the ultrasound probe according to Embodiment 1, respectively, and the ultrasound probe of Embodiment 2 operates as connected to the diagnostic apparatus body 10 of FIG. 2 through wireless communication, as is the case with the ultrasound probe of Embodiment 1. The wireless communication board 44, the battery 45 and the transmission/reception board 46, however, are positioned in the housing 43 differently from the wireless communication board 4, the battery 5 and the transmission/reception board 6 of Embodiment 1, respectively.

In other words, the battery 45 is positioned not on a centerline of arrangement C2 of the transducer array in the transducer unit 47 but away from the centerline C2 toward the puncture needle insertion port 48 of the puncture guide 42. As a result, the wireless communication board 44 and the transmission/reception board 46 are each positioned opposite to the puncture needle insertion port 48 with respect to the centerline C2.

In the top face of the puncture guide 42, the weights W are embedded in positions each opposite to the battery 45 with respect to the centerline of arrangement C2 of the transducer array so that one weight W may be located on each side of the direction of arrangement of the transducer array.

The ultrasound probe of the structure as above has a center of gravity G2 lying between the centerline of arrangement C2 of the transducer array and the puncture needle insertion port 48 of the puncture guide 42 in the direction of arrangement of the transducer array and, at the same time, being almost in accordance with the centerline of arrangement C2 of the transducer array in the direction orthogonal to the direction of arrangement of the transducer array when an ultrasound transmission/reception surface of the transducer array is in a horizontal posture.

The center of gravity G2 is at a height under H2/2, one half the height of the housing 43 which is H2, when the ultrasound transmission/reception surface of the transducer array is in a horizontal posture.

Even though the battery 45 is positioned in the probe body 41 away from the centerline C2 of the transducer array in the direction of arrangement of the transducer array, the ultrasound probe according to Embodiment 2 thus has the center of gravity G2 which is similar to the center of gravity G1 of Embodiment 1 in that it lies between the centerline of arrangement C2 of the transducer array and the puncture needle insertion port 48 of the puncture guide 42 in the direction of arrangement of the transducer array and, at the same time, is almost in accordance with the centerline of arrangement C2 of the transducer array in the direction orthogonal to the direction of arrangement of the transducer array. Consequently, an operator is able to press the puncture guide 42 in a stable posture against a subject, and conduct an ultrasonic diagnosis while performing a puncturing maneuver accurately and easily.

### Embodiment 3

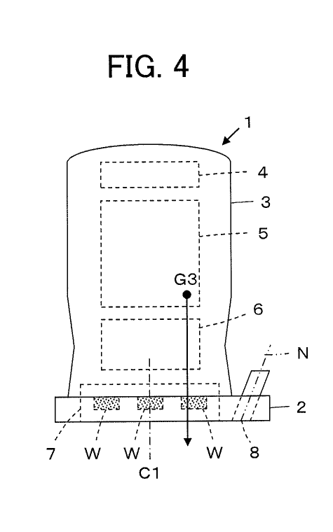

In Embodiment 1, the weights W are embedded in the top face of the puncture guide 2 so that one weight W may be located on each side of the transducer array, although the weights W are not limited in number. As shown in FIG. 4, for instance, a plurality of weights W may be arranged on each side of the transducer array. Also in that case, a center of gravity G3 of the prove lies between the centerline of arrangement C1 of the transducer array and the puncture needle insertion port 8 of the puncture guide 2 in the direction of arrangement of the transducer array and, at the same time, is almost in accordance with the centerline of arrangement C1 of the transducer array in the direction orthogonal to the direction of arrangement of the transducer array.

The same applies to Embodiment 2, that is to say, a plurality of weights W may be arranged on each side of the transducer array of Embodiment 2.

### Embodiment 4

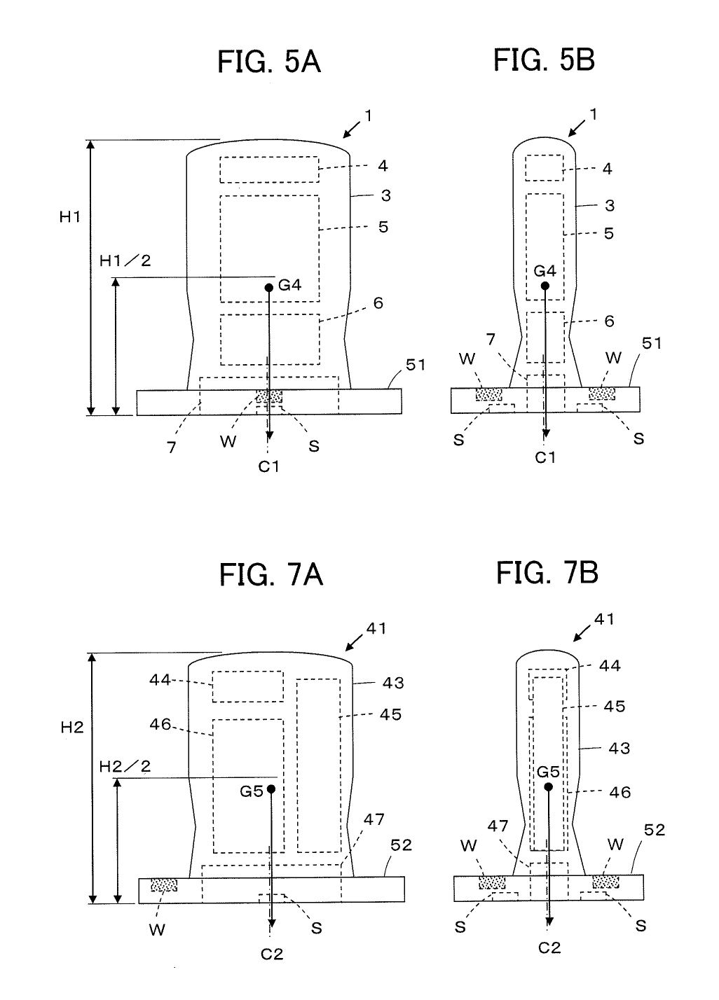

FIGS. 5A and 5B show an ultrasound probe according to Embodiment 4. The ultrasound probe includes a probe body 1, a pressure plate 51 fixed to the probe body 1, and a pair of weights W mounted on the pressure plate 51.

The probe body 1 is identical to the probe body 1 of FIGS. 1A and 1B which is used in the ultrasound probe according to Embodiment 1.

The pressure plate 51 is a plate-shaped member integrally fixed to a housing 3 of the probe body 1. In the top face of the pressure plate 51, the weights W are embedded so that one weight W may be located on each side of a transducer array in a transducer unit 7 in a direction orthogonal to the direction of arrangement of the transducer array.

The bottom face of the pressure plate 51 constitutes a pressure face to be pressed against the surface of a subject during diagnosis by elasticity imaging, and has a pair of pressure sensors S embedded therein so that one sensor S may be located on each side of the transducer array in the direction orthogonal to the direction of arrangement of the transducer array. By mounting the pressure sensors S on the pressure plate 51 in such a manner as above, it can be determined whether or not the pressure plate 51 is so pressed as to apply a uniform pressure to the subject, and the pressing force can be dispersed equally on both sides of the transducer array.

The ultrasound probe of Embodiment 4 has a center of gravity G4 almost in accordance with the centerline of arrangement C1 of the transducer array in both the direction of arrangement of the transducer array and the direction orthogonal to the direction of arrangement of the transducer array when an ultrasound transmission/reception surface of the transducer array is in a horizontal posture.

With the ultrasound probe having such a structure as above, an operator is able to keep the probe with one hand in a stable posture with respect to a subject and, accordingly, press the probe with ease perpendicularly to the surface of the subject.

The center of gravity G4 is at a height under H1/2, one half the height of the housing 3 which is H1, when the ultrasound transmission/reception surface of the transducer array is in a horizontal posture, so that an operator is able to manipulate the probe in a more stable posture.

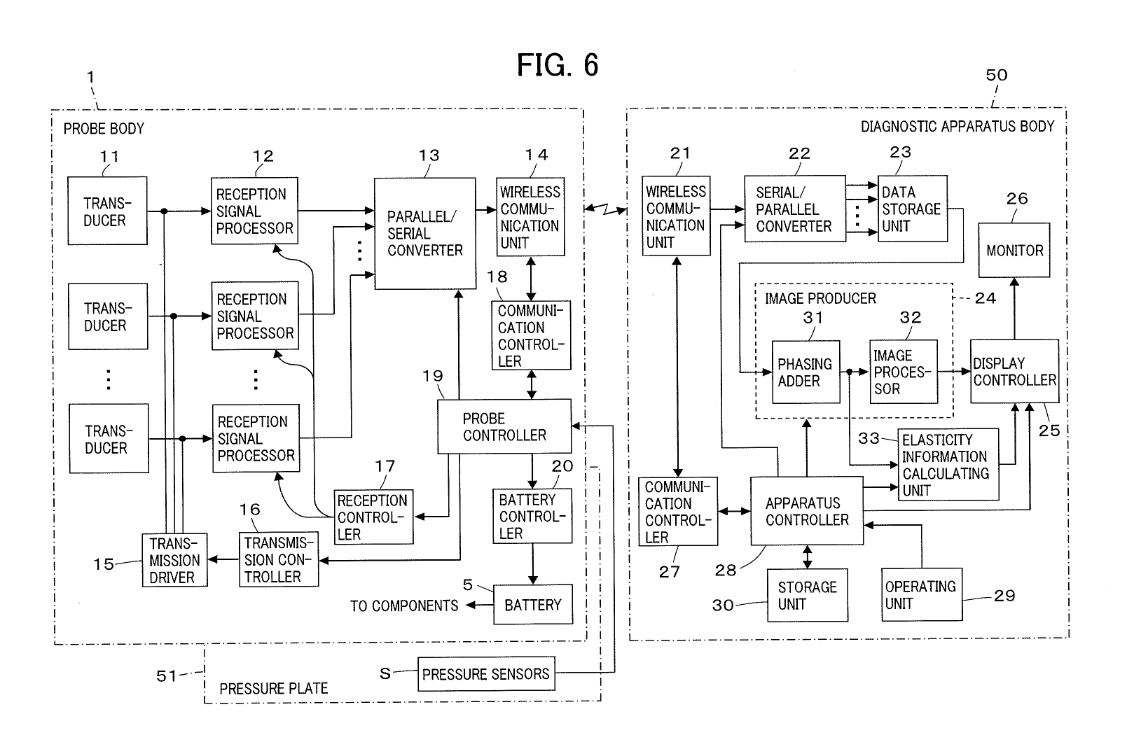

FIG. 6 shows the configuration of an ultrasonic diagnostic apparatus provided with the ultrasound probe according to Embodiment 4. A diagnostic apparatus body 50 is connected to the probe body 1 as an external communication device through wireless communication.

To a probe controller 19 in the probe body 1, the two pressure sensors S embedded in the pressure plate 51 are individually connected in an electrical manner.

The diagnostic apparatus body 50 is the diagnostic apparatus body 10 as shown in FIG. 2 to which an elasticity information calculating unit 33 connected to a phasing adder 31, an apparatus controller 28 and a display controller 25 is newly added, with components other than the unit 33 being the same.

Under the control of the apparatus controller 28, the elasticity information calculating unit 33 calculates information on the elasticity of the region of interest from stress distribution and distortion data according to the sound ray signal as generated by the phasing adder 31 in an image producer 24 and the output signals from the pressure sensors S that are inputted by the apparatus controller 28, and subjects the elasticity information thus obtained to imaging to output it to the display controller 25.

While the elasticity information calculating unit 33 as well as a serial/parallel converter 22, the image producer 24, the display controller 25, a communication controller 27 and the apparatus controller 28 are implemented by a CPU associated with operation programs for giving the CPU instructions on various kinds of processing, the above elements may also be implemented by a digital circuitry.

The ultrasound probe according to Embodiment 4 is not particularly limited in weight. As an example, the ultrasound probe is such that the housing 3 is 40 g, a wireless communication board 4 is 10 g, a battery 5 is 40 g, a transmission/reception board 6 is 30 g, the transducer unit 7 is 20 g, the pressure plate 51 is 40 g, and the weights W are 7 g each in weight.

The ultrasound probe of Embodiment 4 operates as follows.

During a diagnosis by elasticity imaging, an operator initially holds the ultrasound probe having the pressure plate 51 attached thereto with one hand to bring the bottom face of the pressure plate 51 into contact with the surface of a subject, then conducts measurements while pressing the plate 51 against the subject surface. Transducers 11 transmit ultrasonic waves according to the driving signals as supplied from a transmission driver 15 of the prove body 1, and receive ultrasonic echoes from the subject before they output reception signals to corresponding reception signal processors 12, respectively. The sample data as generated by the reception signal processors 12 supplied with the reception signals are converted by a parallel/serial converter 13 into serial data, then transmitted from a wireless communication unit 14 to the diagnostic apparatus body 50 through wireless communication. The sample data as received by a wireless communication unit 21 of the diagnostic apparatus body 50 are converted by the serial/parallel converter 22 into parallel data, and stored in a data storage unit 23. Subsequently, the sample data are read out per frame from the data storage unit 23, and transmitted to the image producer 24. At the same time, the output signals from the pressure sensors S arranged on the lower side of the pressure plate 51 are transmitted to the wireless communication unit 14 through the probe controller 19, then transmitted from the unit 14 to the diagnostic apparatus body 50 through wireless communication. The signals from the sensors S as received by the wireless communication unit 21 of the diagnostic apparatus body 50 are inputted into the elasticity information calculating unit 33 through the apparatus controller 28. In the elasticity information calculating unit 33, information on the elasticity of the region of interest is calculated from the stress distribution and distortion data according to the sound ray signal as generated by the phasing adder 31 in the image producer 24 and the output signals from the pressure sensors S, and the elasticity information thus obtained is subjected to imaging and outputted to the display controller 25. In consequence, an elasticity image is displayed on a monitor 26 by the display controller 25.

By using the ultrasound probe of Embodiment 4, the pressure plate 51 as pressed against a subject can be kept in a stable posture with respect to the subject because the center of gravity G4 of the probe is almost in accordance with the centerline of arrangement C1 of the transducer array in both the direction of arrangement of the transducer array and the direction orthogonal to the direction of arrangement of the transducer array.

Since the weights W are arranged so that one weight W may be located on each side of the transducer array in the direction orthogonal to the direction of arrangement of the transducer array, the tilting in a plane perpendicular to the direction of arrangement of the transducer array, so-called “pitching,” hardly occurs, which also makes it possible to keep the pressure plate 51 in a stable posture, and thereby conduct a diagnosis by elasticity imaging accurately and easily.

Moreover, the weights W embedded in the pressure plate 51 allow the center of gravity G4 to be present at a height under H1/2, one half the height of the housing 3 which is H1, when the ultrasound transmission/reception surface of the transducer array is in a horizontal posture, so that the ultrasound probe is manipulated in a more stable posture.

In Embodiment 4, the weights W are embedded in the top face of the pressure plate 51, although not limitedly. The weights W may be embedded in the bottom face of the pressure plate 51.

### Embodiment 5

FIGS. 7A and 7B show an ultrasound probe according to Embodiment 5. The ultrasound probe includes a probe body 41, a pressure plate 52 fixed to the probe body 41, and a pair of weights W mounted on the pressure plate 52.

The probe body 41 is identical to the probe body 41 of FIGS. 3A and 3B which is used in the ultrasound probe according to Embodiment 2. In other words, a battery 45 is positioned not on a centerline of arrangement C2 of a transducer array in a transducer unit 47 but away from the centerline C2 in the direction of arrangement of the transducer array, and a wireless communication board 44 and a transmission/reception board 46 are each positioned opposite to the battery 45 with respect to the centerline C2.

In the top face of the pressure plate 52, the weights W are embedded in positions each opposite to the battery 45 with respect to the centerline of arrangement C2 of the transducer array so that one weight W may be located on each side of the direction of arrangement of the transducer array.

In the ultrasound probe of Embodiment 5, the battery 45 is positioned away from the centerline of arrangement C2 of the transducer array as shown in FIG. 7A in such a situation that a larger and heavier battery is to be used in a housing 43 with a limited capacity to feed power enough for extended periods of use. The weights W, which are each mounted on the pressure plate 52 opposite to the battery 45 with respect to the centerline C2 so that one weight W may be located on each side of the direction of arrangement of the transducer array, allow a center of gravity G5 of the probe as such to be almost in accordance with the centerline of arrangement C2 of the transducer array in both the direction of arrangement of the transducer array and the direction orthogonal to the direction of arrangement of the transducer array when the ultrasound transmission/reception surface of the transducer array is in a horizontal posture.

Even though the battery 45 is positioned in the probe body 41 away from the centerline C2 of the transducer array in the direction of arrangement of the transducer array, the ultrasound probe according to Embodiment 5 thus has the center of gravity G5 which is similar to the center of gravity G4 of Embodiment 4 in that it is almost in accordance with the centerline of arrangement C2 of the transducer array. Consequently, an operator is able to press the pressure plate 52 in a stable posture against a subject, and conduct a diagnosis by elasticity imaging accurately and easily.

### Embodiment 6

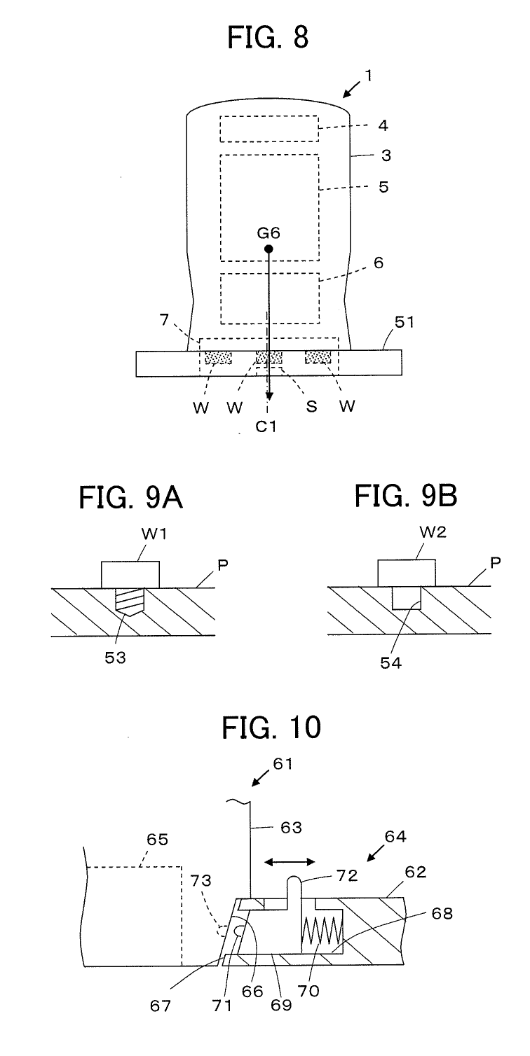

In Embodiment 4, the weights W are embedded in the top face of the pressure plate 51 so that one weight W may be located on each side of the transducer array, although the weights W are not limited in number. As shown in FIG. 8, for instance, a plurality of weights W may be arranged on each side of the transducer array. Also in that case, a center of gravity G6 of the prove is almost in accordance with the centerline of arrangement C1 of the transducer array in both the direction of arrangement of the transducer array and the direction orthogonal to the direction of arrangement of the transducer array.

The same applies to Embodiment 5, that is to say, a plurality of weights W may be arranged on each side of the transducer array of Embodiment 5.

### Embodiment 7

In each of Embodiments 1 through 6 as above, the weights W are embedded in the top face of a flat part P of the puncture guide 2 or 42 or the pressure plate 51 or 52 in order to bring the prove weight into balance. The weights W may be replaced by weights detachable from the flat part P.

As shown in FIG. 9A, for instance, a weight W1 is usable which is screwed into a female screw 53 formed in the top face of the flat part P. The weight W1 is not only fixed to the flat part P securely but is readily detachable from the flat part P, making it possible to modify the weight to be loaded on the flat part P as appropriate to the conditions for a puncturing maneuver or a diagnosis by elasticity imaging.

A weight W2 shown in FIG. 9B, whose lower half is inserted in a recess 54 formed in the top face of the flat part P, may also be used. The weight W2 in such form can be attached to and detached from the flat part P more easily.

### Embodiment 8

In the ultrasound probe according to Embodiment 1, the puncture guide 2 is integrally fixed to the housing 3 of the probe body 1, although the present invention is not limited to such a configuration. The ultrasound probe of the present invention may include a puncture guide detachable from the probe body.

As shown in FIG. 10, a probe body 61 and a puncture guide 62 of an ultrasound probe according to Embodiment 8 are fabricated independently of each other, and the prove includes an adapter 64 for allowing the puncture guide 62 to be attached to/detached from a housing 63 of the probe body 61.

The housing 63 of the probe body 61 has a lower portion tapered down toward the bottom of the housing 63 where an ultrasound transmission/reception surface of a transducer array in a transducer unit 65 is bared, with a periphery 66 of the lower portion being tilted inside, accordingly.

The puncture guide 62 has an opening 67 in which the lower portion of the probe body 61 with the tilted periphery 66 is to be inserted, and a groove 68 communicating with the opening 67. The adapter 64 includes an anchor 69 slidably inserted in the groove 68 of the puncture guide 62, and a spring 70 for urging the anchor 69 toward the opening 67 of the puncture guide 62. On the anchor 69, a protrusion 71 directed toward the opening 67 of the puncture guide 62, and a knob 72 projecting out of the groove 68 through the top face of the puncture guide 62 are formed. On the other hand, a recess 73 is formed in the tilted periphery 66 of the probe body 61 in response to the protrusion 71 of the anchor 69.

The puncture guide 62 is attached to the probe body 61 by inserting the lower portion of the probe body 61 in the opening 67 of the puncture guide 62. As the lower portion of the probe body 61 is inserted into the opening 67 of the puncture guide 62, the anchor 69 is temporarily pushed into the groove 68 by the tilted periphery 66 of the probe body 61. When the recess 73 of the probe body 61 reaches the same level as the protrusion 71 on the anchor 69, the anchor 69 is urged by the spring 70 toward the probe body 61 so as to engage the protrusion 71 on the anchor 69 with the recess 73 of the probe body 61, with the attachment of the puncture guide 62 to the probe body 61 being thus completed.

The puncture guide 62 can be detached from the probe body 61 by manipulating the knob 72 to move the anchor 69 away from the probe body 61 and thereby disengaging the protrusion 71 on the anchor 69 and the recess 73 of the probe body 61 from each other.

If a normal ultrasonic diagnosis involving no puncturing maneuvers is to be conducted with the ultrasound probe as above, the puncture guide 62 will readily be detached from the probe body 61 so as to conduct the diagnosis using solely the probe body 61.

Also in each of the ultrasound probes of Embodiments 2 and 3, the puncture guide may be made detachable from the probe body.

Moreover, in each of the ultrasound probes of Embodiments 4 through 6, the pressure plate may be made detachable from the probe body.

Although the diagnostic apparatus body 10 or 50 is connected to the probe body 1 as an external communication device in each of the above embodiments, a repeater may be used as an external communication device. In this case, the probe body 1 is connected to the diagnostic apparatus body 10 or 50 via a repeater through wireless communication.

What is claimed is:

1. An ultrasound probe, comprising:
a transducer array having an ultrasound transmission/reception surface;
a transmission/reception board including a transmission/reception circuit for the transducer array;
a wireless communication board having a wireless communication circuit mounted thereon for wireless communication with an external communication device;
a battery for supplying power to the transmission/reception circuit and the wireless communication circuit;
a housing for containing the transducer array, the transmission/reception board, the wireless communication board and the battery; and
a puncture guide fixed to the housing and having a puncture needle insertion port located on a line extending in a direction of arrangement of the transducer array,
wherein when the ultrasound transmission/reception surface of the transducer array is in a horizontal posture, the probe has a center of gravity lying between a center of arrangement of the transducer array and the puncture needle insertion port of the puncture guide in the direction of arrangement of the transducer array and, at the same time, being almost in accordance with a centerline of arrangement of the transducer array in a direction orthogonal to the direction of arrangement of the transducer array.

2. The ultrasound probe according to claim 1, further comprising a plurality of weights mounted on the puncture guide so that they may be located on both sides of the transducer array in the direction orthogonal to the direction of arrangement of the transducer array.

3. The ultrasound probe according to claim 2, wherein the weights are each mounted on the puncture guide in a detachable manner.

4. The ultrasound probe according to claim 1, wherein the battery is positioned on the centerline of arrangement of the transducer array.

5. The ultrasound probe according to claim 1, wherein the battery is positioned away from the centerline of arrangement of the transducer array in the direction of arrangement of the transducer array.

6. The ultrasound probe according to claim 5, wherein the weights are positioned opposite to the battery with respect to the centerline of arrangement of the transducer array.

7. The ultrasound probe according to claim 1, wherein the center of gravity is at a height under one half a height of the housing when the ultrasound transmission/reception surface of the transducer array is in a horizontal posture.

8. The ultrasound probe according to claim 1, wherein the puncture guide is detachable from the housing.

9. An ultrasound probe, comprising:
a transducer array having an ultrasound transmission/reception surface;
a transmission/reception board including a transmission/reception circuit for the transducer array;
a wireless communication board having a wireless communication circuit mounted thereon for wireless communication with an external communication device;
a battery for supplying power to the transmission/reception circuit and the wireless communication circuit;
a housing for containing the transducer array, the transmission/reception board, the wireless communication board and the battery; and
a pressure plate fixed to the housing and having a pressure face almost flush with the ultrasound transmission/reception surface of the transducer array,
wherein when the ultrasound transmission/reception surface of the transducer array is in a horizontal posture, the probe has a center of gravity almost in accordance with a centerline of arrangement of the transducer array in both a direction of arrangement of the transducer array and a direction orthogonal to the direction of arrangement of the transducer array.

10. The ultrasound probe according to claim 9, further comprising a plurality of weights mounted on the pressure plate so that they may be located on both sides of the transducer array in the direction orthogonal to the direction of arrangement of the transducer array.

11. The ultrasound probe according to claim 9, wherein the weights are each mounted on the pressure plate in a detachable manner.

12. The ultrasound probe according to claim 9, wherein the battery is positioned on the centerline of arrangement of the transducer array.

13. The ultrasound probe according to claim 9, wherein the battery is positioned away from the centerline of arrangement of the transducer array in the direction of arrangement of the transducer array.

14. The ultrasound probe according to claim 13, wherein the weights are positioned opposite to the battery with respect to the centerline of arrangement of the transducer array.

15. The ultrasound probe according to claim 9, wherein the center of gravity is at a height under one half a height of the housing when the ultrasound transmission/reception surface of the transducer array is in a horizontal posture.

16. The ultrasound probe according to claim 9, further comprising a plurality of pressure sensors mounted on the pressure plate so that they may be located on both sides of the transducer array in the direction orthogonal to the direction of arrangement of the transducer array.

17. The ultrasound probe according to claim 9, wherein the pressure plate is detachable from the housing.
